(19) United States
(12) Patent Application Publication
(10) Pub. No.: US 2003/0230383 A1
(43) Pub. Date: Dec. 18, 2003

(54) Method and apparatus for bonded fluidic circuit for optical bio-disc

(76) Inventor: Glenn Sasaki, EI Cajon, CA (US)

Correspondence Address: COUDERT BROTHERS LLP, 333 SOUTH HOPE STREET, 23RD FLOOR, LOS ANGELES, CA, 90071, US

(21) Appl. No.: 10/205,011

(22) Filed: Jul. 24, 2002

Related U.S. Application Data
(60) Provisional application No. 60/307,488, filed on Jul. 24, 2001.

(51) Int. Cl. C12M 1/34; B29C 67/00; C09J 5/06; C12Q 1/00

(52) U.S. Cl. 156/321; 435/4; 435/287.2

(57) ABSTRACT

Embodiments of the present invention are directed to a method and apparatus for bonded fluidic circuit for an optical bio-disc. In one embodiment of the present invention, a bio-disc is formed using at least two discs. In one embodiment, a shim is used to bond the two discs. In another embodiment, ultra-violet (UV) cured adhesives are used to bond the two discs. In yet another embodiment, the two discs are welded together using ultrasonic energy. In one embodiment, flash chambers containing a fluid are included in the fluidic circuit. During use of the bio-disc, the flash chambers are heated using a laser, causing the fluid to expand and/or vaporize. The expansion of the fluid in the flash chamber is used to propel the sample fluid through the fluidic circuit as desired.

### RELATED APPLICATION INFORMATION

This application claims the benefit of United States Provisional Patent Application, serial No. 60/307,488, filed Jul. 24, 2001, entitled, “Bonded Fluidic Circuit for Optical Bio-Disc,” the disclosure of which is hereby incorporated by reference.

### BACKGROUND OF THE INVENTION

1. Field of the Invention

The present invention relates to the field of optical bio-discs, and in particular to a method and apparatus for bonded fluidic circuit for an optical bio-disc.

2. Background Art

A bio-disc is similar to a CD or DVD; however, instead of storing audio/visual or other data, a bio-disc may be used to diagnose certain ailments inside or outside of a doctor's office. Because of the content deposited in on bio-discs, they must meet rigorous safety standards that make manufacture of the discs difficult.

Bio-discs may be utilized in home medical testing ranging from pregnancy tests to testing for cancer or the Ebola virus. Typically, a test sample (e.g., urine or blood) is placed in a receptacle of the bio-disc and is tested by various means. For example, the fluid may be forced past reactive regions in the disc. Then, the fluid or the regions can be analyzed to determine the test results.

In bio-discs, fluid flow is driven by centrifugal force. As the disc spins, the fluid is forced towards the outer-most parts of the disc. However, this limits configurations of the bio-discs to ones where the fluid never moves closer to the inner-most parts of the disc.

Because bio-discs can rotate at very high speeds (e.g., up to 13,000 RPM), it's possible that any fluid placed in a bio-disc could aerosolize. This could lead to catastrophic results if the fluid is infected with a harmful infectious disease. The problem is compounded by the fact that typically, a bio-disc reader (e.g., a standard CD drive) is typically air-cooled by a fan system that will further disperse the infectious material. Thus, a bio-disc typically has channels that are enclosed between two discs. However, bonding the two discs securely is difficult to accomplish without damaging the reactive substances or other aspects of the bio-disc.

### SUMMARY OF THE INVENTION

Embodiments of the present invention are directed to a method and apparatus for bonded fluidic circuit for an optical bio-disc. In one embodiment of the present invention, a bio-disc is formed using at least two discs. The upper disc contains grooves (or channels) to accommodate fluid flow, and the lower disc contains the wobble groove and gold coating on its upper surface. The two discs are bonded together such that there is no gap between the discs, except where channels exist.

One embodiment of the present invention is employed in an optical bio-disc, which is a modified optical disc similar to CD, CD-R, CD-RW, DVD or equivalents widely available in the market today. An optical bio-disc contains fluidic flow chamber on the disc surface for housing assay solution and magnetic beads. A bio-disc drive assembly is employed to rotate the disc, read and process any encoded information stored on the disc, and analyze the cell capture zones in the flow chamber of the bio-disc. The bio-disc drive is provided with a motor for rotating the bio-disc, a controller for controlling the rate of rotation of the disc, a processor for processing return signals from the disc, and analyzer for analyzing the processed signals. The rotation rate is variable and may be closely controlled both as to speed and time of rotation. The bio-disc may also be utilized to write information to the bio-disc either before or after the test material in the flow chamber and target zones is interrogated by the read beam of the drive and analyzed by the analyzer. The bio-disc may include encoded information for controlling the rotation of the disc, providing processing information specific to the type of immunotyping assay to be conducted and for displaying the results on a monitor associated with the bio-drive.

In one embodiment, a shim (a thin material) is used to bond the two discs. In one embodiment, the shim is a pressure-activated adhesive. In another embodiment, the shim is a heat-activated adhesive wherein the heat level necessary for activation is lower than the heat level at which any of the fluidic channels or reactive areas are damaged. In one embodiment, the channels are cut into the shim. Thus, the thickness of the shim determines the minimum height of the grooves. In another embodiment, a raised groove in the upper disc is used to form a channel with a height less than the thickness of the shim. In another embodiment, grooves in the upper disc are combined with channels in the shim to produce deeper grooves. In still other embodiments, fluid chambers, reservoirs and other fluidic circuit components are cut out of the upper disc.

In another embodiment, ultra-violet (UV) cured adhesives are used to bond the two discs. A low viscosity (e.g., less than 100 cp) adhesive is applied to the surface of the upper disc. In one embodiment, the adhesive is sprayed on. In embodiments where the adhesive does not interfere with operation of the reactive areas or with the analysis of the results, the adhesive may be sprayed over the grooves as well. In other embodiments, a mask is used to prevent the adhesive from covering the grooves. In another embodiment, the adhesive is stamped on. In yet another embodiment, the adhesive is rolled on. Once the discs are properly positioned, UV light is used to cure the bond. In one embodiment, the wavelength of the UV light is selected so that the adhesive cures, but no damage is done to the fluidic circuit. In another embodiment, the intensity of the UV light is limited to prevent damage to the fluidic circuit. In yet another embodiment, the length of the UV exposure is limited to prevent damage to the fluidic circuit.

In another embodiment, the plastic areas of the upper disc that are not part of the fluidic circuit are made hydrophilic (e.g., using plasma etching or some other surface modification technique). Then, a hydrophilic adhesive is applied. Thus, the adhesive coats the non-circuit portions of the disc without interfering with the circuit portions of the disc. Similarly, in yet another embodiment, the plastic areas of the upper disc that are not part of the fluidic circuit are made hydrophobic. Then, a hydrophobic adhesive is applied.

In another embodiment, plasma etching (or some other surface modification technique) is used to charge the surface of the disc where there is no fluidic circuit. Then, through chemical attachment of the active site, an adhesive is covalently bonded to the surface of the disc.

In various embodiments, the adhesive is sprayed, electrocoated, inkjetted, vacuum deposited, or screen printed using a mask to control where the adhesive is applied. In yet another embodiment, the two discs are welded together using ultrasonic energy.

In one embodiment, flash chambers containing a fluid are included in the fluidic circuit. The fluid (e.g., water) will not interfere with the reactions taking place during use of the bio-disc. In one embodiment, fluid in a flash chamber actually assists the reactions occurring during use of the bio-disc. During use of the bio-disc, the flash chambers are heated using a laser, causing the fluid to expand and/or vaporize. The expansion of the fluid in the flash chamber is used to propel the sample fluid through the fluidic circuit as desired. Thus, the sample fluid can be made to flow towards the inner-most parts of the bio-disc and circuit designs are no longer limited to only centrifugal force driven designs.

The present invention is also directed to bio-discs, bio-drives, and related methods. This invention or different aspects thereof may be readily implemented in, adapted to, or employed in combination with the discs, assays, and systems disclosed in the following commonly assigned and co-pending patent applications: U.S. patent application Ser. No. 09/378,878 entitled “Methods and Apparatus for Analyzing Operational and Non-operational Data Acquired from Optical Discs” filed Aug. 23, 1999; U.S. Provisional Patent Application Serial No. 60/150,288 entitled “Methods and Apparatus for Optical Disc Data Acquisition Using Physical Synchronization Markers” filed Aug. 23, 1999; U.S. patent application Ser. No. 09/421,870 entitled “Trackable Optical Discs with Concurrently Readable Analyte Material” filed Oct. 26, 1999; U.S. patent application Ser. No. 09/643,106 entitled “Methods and Apparatus for Optical Disc Data Acquisition Using Physical Synchronization Markers” filed Aug. 21, 2000; U.S. patent application Ser. No. 09/999,274 entitled “Optical Bio-discs with Reflective Layers” filed Nov. 15, 2001; U.S. patent application Ser. No. 09/988,728 entitled “Methods And Apparatus For Detecting And Quantifying Lymphocytes With Optical Bio-discs” filed Nov. 20, 2001; U.S. patent application Ser. No. 09/988,850 entitled “Methods and Apparatus for Blood Typing with Optical Bio-discs” filed Nov. 19, 2001; U.S. patent application Ser. No. 09/989,684 entitled “Apparatus and Methods for Separating Agglutinants and Disperse Particles” filed Nov. 20, 2001; U.S. patent application Ser. No. 09/997,741 entitled “Dual Bead Assays Including Optical Bio-discs and Methods Relating Thereto” filed Nov. 27, 2001; U.S. patent application Ser. No. 09/997,895 entitled “Apparatus and Methods for Separating Components of Particulate Suspension” filed Nov. 30, 2001; U.S. patent application Ser. No. 10/005,313 entitled “Optical Discs for Measuring Analytes” filed Dec. 7, 2001; U.S. patent application Ser. No. 10/006,371 entitled “Methods for Detecting Analytes Using Optical Discs and Optical Disc Readers” filed Dec. 10, 2001; U.S. patent application Ser. No. 10/006,620 entitled “Multiple Data Layer Optical Discs for Detecting Analytes” filed Dec. 10, 2001; U.S. patent application Ser. No. 10/006,619 entitled “Optical Disc Assemblies for Performing Assays” filed Dec. 10, 2001; U.S. patent application Ser. No. 10/020,140 entitled “Detection System For Disc-Based Laboratory And Improved Optical Bio-Disc Including Same” filed Dec. 14, 2001; U.S. patent application Ser. No. 10/035,836 entitled “Surface Assembly For Immobilizing DNA Capture Probes And Bead-Based Assay Including Optical Bio-discs And Methods Relating Thereto” filed Dec. 21, 2001; U.S. patent application Ser. No. 10/038,297 entitled “Dual Bead Assays Including Covalent Linkages For Improved Specificity And Related Optical Analysis Discs” filed Jan. 4, 2002; U.S. patent application Ser. No. 10/043,688 entitled “Optical Disc Analysis System Including Related Methods For Biological and Medical Imaging” filed Jan. 10, 2002; and U.S. Provisional Application Serial No. 60/348,767 entitled “Optical Disc Analysis System Including Related Signal Processing Methods and Software” filed Jan. 14, 2002. All of these applications are herein incorporated by reference in their entireties. They thus provide background and related disclosure as support hereof as if fully repeated herein.

### BRIEF DESCRIPTION OF THE DRAWINGS

These and other features, aspects and advantages of the present invention will become better understood with regard to the following description, appended claims and accompanying drawings where:

FIG. 1 is a block diagram of a cross-section view of a portion of two discs of a bio-disc in accordance with one embodiment of the present invention;

FIG. 2 is a flow diagram of the process of forming a bio-disc using a shim in accordance with one embodiment of the present invention;

FIG. 3 is a flow diagram of the process of forming a bio-disc using a UV cured adhesive in accordance with one embodiment of the present invention;

FIG. 4 is a flow diagram of the process of applying adhesive to a disc in accordance with one embodiment of the present invention; and

FIG. 5 is a block diagram of a fluidic circuit of a bio-disc in accordance with one embodiment of the present invention.

### DETAILED DESCRIPTION OF THE INVENTION

The invention is a method and apparatus for bonded fluidic circuit for an optical bio-disc. In the following description, numerous specific details are set forth to provide a more thorough description of embodiments of the invention. It is apparent, however, to one skilled in the art, that the invention may be practiced without these specific details. In other instances, well known features have not been described in detail so as not to obscure the invention.

In one embodiment of the present invention, a bio-disc is formed using at least two discs. The upper disc contains grooves (or channels) to accommodate fluid flow, and the lower disc contains the wobble groove and gold coating on its upper surface. The two discs are bonded together such that there is no gap between the discs, except where channels exist.

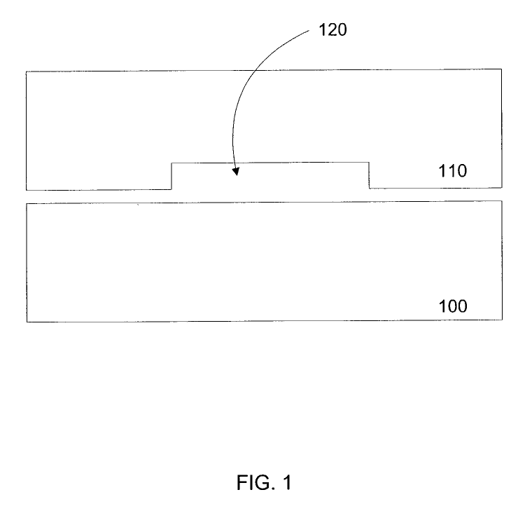

FIG. 1 illustrates a cross-section view of a portion of two discs of a bio-disc in accordance with one embodiment of the present invention. The bottom disc 100 contains the wobble groove and a gold coating on its upper surface. The top disc 110 contains a groove 120 used to form a channel. In one embodiment, groove 120 is 1 mm wide and 100 microns deep.

Shim Bonding

In one embodiment, a shim (a thin material) is used to bond the two discs. In one embodiment, the shim is a pressure-activated adhesive. In another embodiment, the shim is a heat-activated adhesive wherein the heat level necessary for activation is lower than the heat level at which any of the fluidic channels or reactive areas are damaged. In one embodiment, the channels are cut into the shim. Thus, the thickness of the shim determines the minimum height of the grooves. In another embodiment, a raised groove in the upper disc is used to form a channel with a height less than the thickness of the shim. In another embodiment, grooves in the upper disc are combined with channels in the shim to produce deeper grooves. In still other embodiments, fluid chambers, reservoirs and other fluidic circuit components are cut out of the upper disc.

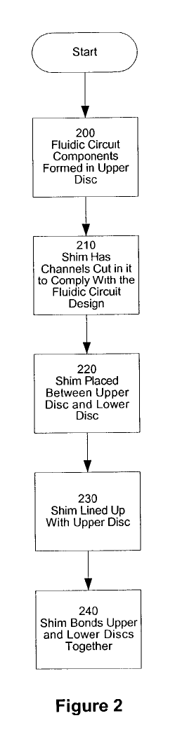

FIG. 2 illustrates the process of forming a bio-disc using a shim in accordance with one embodiment of the present invention. At block 200, fluidic circuit components are formed in an upper disc. At block 210, a shim has channels cut in it to comply with the fluidic circuit design. At block 220, the shim is placed between the upper disc and a lower disc. At block 230, the shim is lined up with the upper disc. At block 240, the shim bonds the upper and lower discs together. In one embodiment, the bonding in block 240 involves the application of heat. In another embodiment, the bonding is accomplished by pressing the upper and lower discs towards each other with the shim in the middle.

Adhesive Bonding

In another embodiment, ultra-violet (UV) cured adhesives are used to bond the two discs. A low viscosity (e.g., less than 100 cp) adhesive is applied to the surface of the upper disc. In another embodiment, the adhesive is applied to the lower disc. In one embodiment, the adhesive is sprayed on. In embodiments where the adhesive does not interfere with operation of the reactive areas or with the analysis of the results, the adhesive may be sprayed over the grooves as well. In other embodiments, a mask is used to prevent the adhesive from covering the grooves. In another embodiment, the adhesive is stamped on. In yet another embodiment, the adhesive is rolled on. Once the discs are properly positioned, UV light is used to cure the bond. In one embodiment, the wavelength of the UV light is selected so that the adhesive cures, but no damage is done to the fluidic circuit. In another embodiment, the intensity of the UV light is limited to prevent damage to the fluidic circuit. In yet another embodiment, the length of the UV exposure is limited to prevent damage to the fluidic circuit.

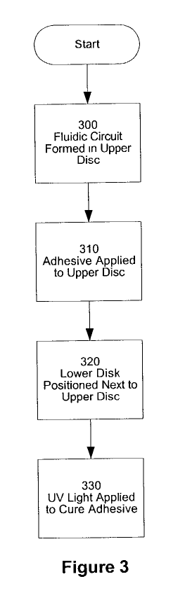

FIG. 3 illustrates the process of forming a bio-disc using a UV cured adhesive in accordance with one embodiment of the present invention. At block 300, the fluidic circuit is formed in an upper disc. At block 310, an adhesive is applied to the upper disc. At block 320, a lower disc is positioned next to the upper disc. At block 330, UV light is applied to cure the adhesive.

Controlling Adhesive Placement

In another embodiment, the plastic areas of the upper disc that are not part of the fluidic circuit are made hydrophilic (e.g., using plasma etching or some other surface modification technique). Then, a hydrophilic adhesive is applied. Thus, the adhesive coats the non-circuit portions of the disc without interfering with the circuit portions of the disc. Similarly, in yet another embodiment, the plastic areas of the upper disc that are not part of the fluidic circuit are made hydrophobic. Then, a hydrophobic adhesive is applied.

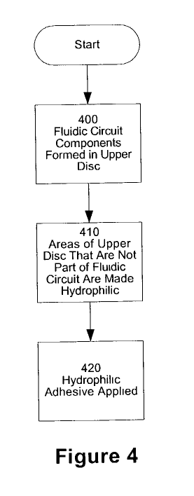

FIG. 4 illustrates the process of applying adhesive to a disc in accordance with one embodiment of the present invention. At block 400, fluidic circuit components are formed in an upper disc. At block 410, areas of the upper disc that are not part of the fluidic circuit are made hydrophilic. At block 420, a hydrophilic adhesive is applied. Thus, the adhesive is attracted to the hydrophilic sections of the upper disc and do not cover or interfere with the fluidic circuit.

In another embodiment, plasma etching (or some other surface modification technique) is used to charge the surface of the disc where there is no fluidic circuit. Then, through chemical attachment of the active site, an adhesive is covalently bonded to the surface of the disc. In various embodiments, the adhesive is sprayed, electrocoated, inkjetted, vacuum deposited, or screen printed using a mask to control where the adhesive is applied. In yet another embodiment, the two discs are welded together using ultrasonic energy.

Controlling Fluid Flow

In one embodiment, flash chambers containing a fluid are included in the fluidic circuit. The fluid (e.g., water) will not interfere with the reactions taking place during use of the bio-disc. In one embodiment, fluid in a flash chamber actually assists the reactions occurring during use of the bio-disc. During use of the bio-disc, the flash chambers are heated using a laser, causing the fluid to expand and/or vaporize. The expansion of the fluid in the flash chamber is used to propel the sample fluid through the fluidic circuit as desired. Thus, the sample fluid can be made to flow towards the inner-most parts of the bio-disc and circuit designs are no longer limited to only centrifugal force driven designs.

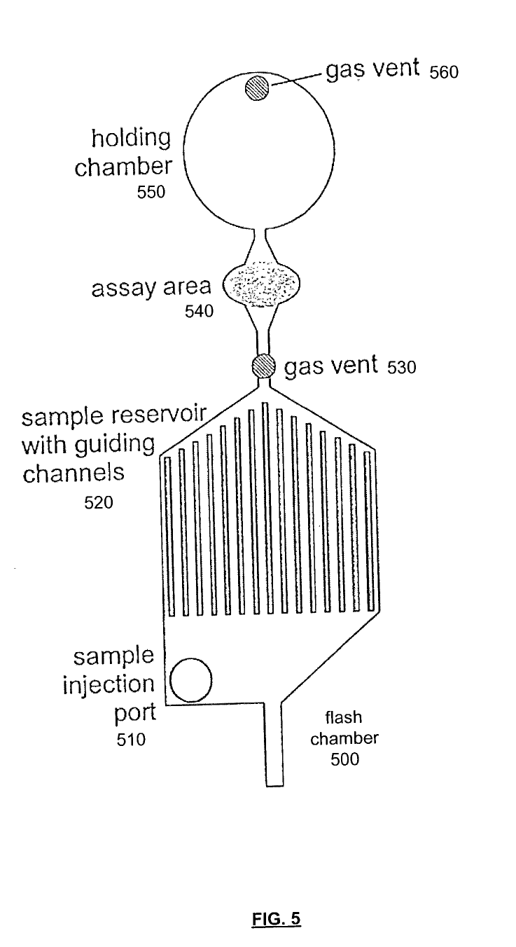

FIG. 5 illustrates a fluidic circuit of a bio-disc in accordance with one embodiment of the present invention. The fluidic circuit consists of a flash chamber 500, sample injection port 510 on a sample reservoir 520, a first gas vent 530, an assay area 540, a holding chamber 550, and a second gas vent 560. After the sample is placed in sample reservoir 520 through sample injection port 510, a laser heats the fluid in flash chamber 500. As the fluid in the chamber vaporizes, the resulting bubble forces the sample past first gas vent 530 and into assay area 540 where the desired reactions take place before the sample passes into holding chamber 550.

In one embodiment, the fluidic circuit of FIG. 5 is positioned on the bio-disc such that the flash chamber is near the outer-most part of the disc and the holding chamber is nearer the inner-most part of the disc. Thus, there is more space available to make a large sample reservoir. Additionally, centrifugal force can be applied by spinning the bio-disc to force the sample back past the assay and into the reservoir again. Heat could again be applied to the flash chamber to force the sample back into the holding chamber. Thus, a sample can be exposed to an assay multiple times before the results are analyzed.

Washing

In one embodiment, the assay area has the ability to capture desired substances (e.g., white blood cells) from the sample. However, before this captured material is analyzed, the fluidic circuit is washed using a washing fluid to remove unwanted substances from the analysis area. Thus, the unwanted substances do not interfere with the analysis. In one embodiment, the washing fluid removes unwanted substances by simply physically pushing them away from the analysis area. In another embodiment, the washing fluid contains chemicals that interact with the unwanted substances to facilitate their removal.

Thus, a method and apparatus for bonded fluidic circuit for an optical bio-disc is described in conjunction with one or more specific embodiments. The invention is defined by the following claims and their full scope and equivalents.

What is claimed is:

1. A method of forming a bio-disc comprising:
creating channels in a shim; and
bonding an upper disc to a lower disc using said shim.

2. The method of claim 1 wherein said step of bonding comprises:
heating said shim.

3. The method of claim 1 wherein said step of bonding comprises:
pressing said upper disc against said shim; and
pressing said lower disc against said shim.

4. The method of claim 1 further comprising:
forming a fluidic circuit component in said upper disc.

5. A method of forming a bio-disc comprising:
forming a fluidic circuit in an upper disc; and
bonding said upper disc to a lower disc using an adhesive.

6. The method of claim 5 further comprising:
applying said adhesive to said upper disc.

7. The method of claim 6 further comprising:
modifying a region of said upper disc to make said region hydrophilic,
wherein said adhesive is hydrophilic.

8. The method of claim 6 further comprising:
modifying a region of said upper disc to make said region hydrophobic,
wherein said adhesive is hydrophobic.

9. The method of claim 6 further comprising:
charging a region of said upper disc; and
covalently bonding said adhesive to said region.

10. The method of claim 6 further comprising:
placing a mask on said upper disc before said adhesive is applied; and
removing said mask after said adhesive is applied.

11. The method of claim 5 further comprising:
curing said adhesive using ultra-violet light.

12. A method of controlling fluid flow in a bio-disc comprising:
heating a first fluid in a fluidic circuit with a laser; and
displacing a second fluid in said fluidic circuit wherein said second fluid is displaced by the expansion of said first fluid caused by said step of heating.

13. The method of claim 12 wherein said step of displacing causes said second fluid to move through said fluidic circuit closer to a center of said bio-disc;

14. The method of claim 13 further comprising:
spinning said bio-disc to cause said second fluid to move away from said center through said fluidic circuit using centrifugal force.

15. The method of claim 12 further comprising:
washing said fluidic circuit with a third fluid wherein said step of washing removes an unwanted substance from an analysis region of said fluidic circuit.

16. A bio-disc comprising:
a shim wherein a channel is formed in said shim;
an upper disc; and
a lower disc wherein said upper disc is bonded to said lower disc using said shim.

17. The bio-disc of claim 16 wherein heat is applied to said shim to bond said upper disc to said lower disc.

18. The bio-disc of claim 16 wherein said upper disc and said lower disc are pressed against said shim to bond said upper disc to said lower disc.

19. The bio-disc of claim 16 further comprising:
a fluidic circuit component formed in said upper disc.

20. A bio-disc comprising:
an upper disc;
a fluidic circuit in said upper disc;
a lower disc; and
an adhesive configured to bond said upper disc to a lower disc.

21. The bio-disc of claim 20 wherein said adhesive is applied to said upper disc.

22. The bio-disc of claim 21 wherein a region of said upper disc is modified to make said region hydrophilic and wherein said adhesive is hydrophilic.

23. The bio-disc of claim 21 wherein a region of said upper disc is modified to make said region hydrophobic and wherein said adhesive is hydrophobic.

24. The bio-disc of claim 21 wherein a region of said upper disc is charged and wherein said adhesive is covalently bonded to said region.

25. The bio-disc of claim 21 further comprising:
a mask wherein said mask is placed on said upper disc before said adhesive is applied and wherein said mask is removed after said adhesive is applied.

26. The bio-disc of claim 20 wherein said adhesive is cured using ultraviolet light.

27. A fluid flow control system for a bio-disc comprising:
a fluidic circuit;
a laser configured to heat a first fluid in said fluidic circuit causing it to expand wherein a second fluid in said fluidic circuit is displaced by the expansion of said first fluid.

28. The fluid flow control system for a bio-disc of claim 27 wherein said second fluid moves through said fluidic circuit closer to a center of said bio-disc;

29. The fluid flow control system for a bio-disc of claim 28 further comprising:
a rotation system configured to spin said bio-disc to cause said second fluid to move away from said center through said fluidic circuit using centrifugal force.

30. The fluid flow control system for a bio-disc of claim 27 further comprising:
a washing system configured to wash said fluidic circuit with a third fluid wherein said third fluid removes an unwanted substance from an analysis region of said fluidic circuit.
